(19) United States
(12) Patent Application Publication
(10) Pub. No.: US 2026/0218696 A1
(43) Pub. Date: Jul. 30, 2026

(54) GAMMA STABLE REINFORCED PUMP TUBING

(71) Applicant: W. L. Gore & Associates, Inc., Newark, DE (US)

(72) Inventors: Ronald D. Gott, Newark, DE (US); Shannon L. Levy, Newark, DE (US); Guy A. Sbriglia, Newark, DE (US)

(73) Assignee: W. L. Gore & Associates, Inc., Newark, DE (US)

(21) Appl. No.: 19/147,482

(22) PCT Filed: Dec. 8, 2023
(86) PCT No.: PCT/US2023/083155

§ 371 Date: Jul. 11, 2025

Related U.S. Application Data
(60) Provisional application No. 63/438,366, filed on Jan. 11, 2023.

(51) Int. Cl. F04B 43/00; B32B 1/08; B32B 27/06; B32B 27/32; F16L 11/04

(52) U.S. Cl. CPC F04B 43/0072; B32B 1/08; B32B 27/065; B32B 27/32; F16L 11/045; B32B 2250/05; B32B 2250/22; B32B 2260/048; B32B 2266/025; B32B 2307/546; B32B 2597/00

(57) ABSTRACT

A composite tube having at least one porous polyethylene layer imbibed with at least one elastomer to form a composite layer having an elastic modulus less than 40 MPa and a primary melt peak temperature less than 135° C. is disclosed. The at least one elastomer is at least partially imbibed through a thickness of the porous polyethylene layer. The porous polyethylene layer may include expanded polyethylene (ePe), or ultra-high molecular weight expanded polyethylene (eUHMWPE). In some embodiments, at least one elastomer forms a coating on the polyethylene layer. The composite tube may have a volume fraction from about 1% to about 20% and an elastic modulus from about 1 MPa to about 40 MPa. A gamma sterilized polymeric composite tube may have an average pump life from about 80 hours to about 5000 hours. In some embodiments, the composite tube may include a reinforcing film.

### FIELD

The present disclosure relates generally to pump tubing. More specifically, the disclosure relates to a sterilized composite pump tubing that includes an elastomer reinforced with polyethylene.

### BACKGROUND

Silicone elastomers can be fabricated into many forms for use, illustratively, in the medical, electrical, and chemical industries. Articles such as peristaltic pump tubes, pump diaphragms, bellows, baby bottle nipples, wire and cable sheaths, gaskets, and O-rings, for example, are commonly made from silicone elastomers. Many of these articles, moreover, are used in applications that require repeated flexing. For example, peristaltic pumps are used to transport liquids and pastes through an elastomeric tube in which the tube is squeezed between a set of rotating rollers and a fixed pump housing. Silicone elastomers are frequently used for peristaltic pump tubing. Upon repeated flexure, however, the silicone rubber tubing can develop cracks in the side wall and rupture catastrophically or lose restitution leading to flow decay. The problem is exacerbated when pumping fluids at elevated pressures and temperatures, leading to even shorter pump tubing life.

Silicones are a class of inherently flexible polymers with organosilicon-oxygen repeating units which undergo bond rotation with little resistance. As a result, silicones possess excellent low temperature properties; however, their weak intermolecular and intramolecular polymer interactions result in poor tear strength and toughness. As a result, silicone elastomers are often reinforced with either particulate inorganic fillers or soluble silicone resin fillers. Inorganic fillers, such as fumed silica, for example, are known to increase the tensile strength of dimethyl silicones by a factor of ten. Some silicone elastomers are limited to approximately 1,300 psi tensile strength (ASTM D-412) and 250 ppi tear strength (ASTM D-624 die B). Natural rubber, on the other hand, has significantly higher tensile and tear properties; however, it lacks many of the useful silicone elastomer attributes of low temperature flexibility, low dielectric loss, ozone resistance, low extractables, and radiation resistance.

Previously silicones were reinforced with a polymer such as polytetrafluoroethylene (PTFE) or expanded PTFE (ePTFE) for increased durability (see U.S. Pat. No. 6,451,396, to Zumbrum, et al.). However, in instances where gamma irradiation is used as a sterilization technique, for example for sterilizing tubes for single use pump systems, the polymer degrades when exposed to gamma irradiation, resulting in poor pump life. Thus, there is a continuing need for more durable pump tubing, especially in cases where the tubes are sterilized with gamma irradiation (i.e., γ-sterilized).

### SUMMARY

The present disclosure generally relates to a composite pump tubing having an elastomer reinforced with porous polyethylene.

According to a first embodiment (“Embodiment 1”), a composite tube includes a tube wall having at least one porous polyethylene layer, each said porous polyethylene layer being imbibed with at least one elastomer to form a composite layer having an elastic modulus less than 40 MPa and a primary melt peak temperature less than 135° C.

Embodiment 2 is the composite tube of Embodiment 1, wherein the composite tube has an average pump life greater than 80 hours.

Embodiment 3 is the composite tube of Embodiment 1 or 2, wherein the tube wall has a volume fraction from 1% to 20%.

Embodiment 4 is the composite tube of Embodiment 3, wherein the composite layer has an elastic modulus from about 1 MPa to about 40 MPa.

Embodiment 5 is the composite tube of Embodiment 4, wherein the composite layer has an elastic modulus from about 1 MPa to about 24 MPa.

Embodiment 6 is the composite tube of Embodiment 5, wherein the composite layer has an elastic modulus from about 1 MPa to about 13 MPa.

Embodiment 7 is the composite tube of Embodiment 6, wherein the composite layer has an elastic modulus from about 1 MPa to about 7 MPa.

Embodiment 8 is the composite tube of any one of Embodiments 1-7, wherein the porous polyethylene is expanded polyethylene (ePe).

Embodiment 9 is the composite tube of any one of Embodiments 1-8, wherein the porous polyethylene is expanded ultra-high molecular weight polyethylene.

Embodiment 10 is the composite tube of any one of Embodiments 1-9, wherein the elastomer is selected from a silicone, a diene-based rubber, a butyl rubber, fluoroelastomer, a perfluoroelastomer, a perfluoro polyether elastomer, or a thermoplastic elastomer including styrenic, polyether, polyester, polyurethane block copolymers, or combinations thereof.

Embodiment 11 is the composite tube of any one of Embodiments 1-10, wherein the at least one elastomer is at least partially imbibed through a thickness of the porous polyethylene layer.

Embodiment 12 is the composite tube of any one of Embodiments 1-11, wherein the composite tube is γ-sterilized, steam sterilized, autoclave sterilized, EtO sterilized, x-ray sterilized, e-beam sterilized, dry heat sterilized, or cleaned in place (CIP).

Embodiment 13 is the composite tube of any one of Embodiments 1-12, wherein the tube is peristaltic pump tubing.

According to another embodiment (“Embodiment 14”), a peristaltic pump tubing includes a tube wall having at least one porous polymeric layer, each said porous polymeric layer being imbibed with at least one elastomer to form a composite layer having an elastic modulus less than 40 MPa; wherein the peristaltic pump tubing has an average pump life from 300 to 5,000 hours.

Embodiment 15 is the pump tubing of Embodiment 14, wherein the composite layer has a primary melt peak temperature less than 135° C.

Embodiment 16 is the pump tubing of Embodiment 14 or 15, wherein the tube wall has a volume fraction from 1% to 13%.

Embodiment 17 is the pump tubing of Embodiment 16, wherein the composite layer has an elastic modulus from about 1 MPa to about 40 MPa.

Embodiment 18 is the pump tubing of Embodiment 17, wherein the composite layer has an elastic modulus from about 1 MPa to about 24 MPa.

Embodiment 19 is the pump tubing of Embodiment 18, wherein the composite layer has an elastic modulus from about 1 MPa to about 13 MPa.

Embodiment 20 is the pump tubing of Embodiment 19, wherein the composite layer has an elastic modulus from about 1 MPa to about 7 MPa.

Embodiment 21 is the pump tubing of any of Embodiments 14-20, wherein the peristaltic pump tubing is γ-sterilized, steam sterilized, autoclave sterilized, EtO sterilized, x-ray sterilized, e-beam sterilized, dry heat sterilized, or cleaned in place (CIP).

Embodiment 22 is the pump tubing of any of Embodiments 14-21, wherein the at least one elastomer is at least partially imbibed through a thickness of the porous polyethylene layer.

Embodiment 23 is the pump tubing of any of Embodiments 14-22, wherein the elastomer is selected from a silicone, a diene-based rubber, a butyl rubber, fluoroelastomer, a perfluoroelastomer, a perfluoro polyether elastomer, or a thermoplastic elastomer including styrenic, polyether, polyester, polyurethane block copolymers, or combinations thereof.

Embodiment 24 is the pump tubing of Embodiment 14, wherein the at least one porous polymeric layer is selected from porous polyethylene, polypropylene, poly(ether ketone) (PEEK), and copolymers of ethylene and at least one comonomer.

Embodiment 25 is the pump tubing of any of Embodiments 14-24, wherein the at least one porous polymeric layer comprises at least one of expanded polyethylene and ultra-high molecular weight polyethylene.

In yet another embodiment (“Embodiment 26”), a composite tube includes a tube wall having at least one polyethylene layer, each said polyethylene layer being coated with at least one elastomer to form a composite layer having an elastic modulus less than 40 MPa and a primary melt peak temperature less than 135° C.

Embodiment 27 is the composite tube of Embodiment 26, wherein the composite tube has an average pump life greater than about 80 hours.

Embodiment 28 is the composite tube of Embodiment 26 or 27, wherein the tube wall has a volume fraction from 1% to 20%.

Embodiment 29 is the composite tube of Embodiment 28, wherein the composite layer has an elastic modulus from about 1 MPa to about 40 MPa.

Embodiment 30 is the composite tube of Embodiment 29, wherein the composite layer has an elastic modulus from about 1 MPa to about 24 MPa.

Embodiment 31 is the composite tube of Embodiment 30, wherein the composite layer has an elastic modulus from about 1 MPa to about 13 MPa.

Embodiment 32 is the composite tube of Embodiment 31, wherein the composite layer has an elastic modulus from about 1 MPa to about 7 MPa.

Embodiment 33 is the composite tube of any of Embodiments 26-32, wherein the elastomer is selected from a silicone, a diene-based rubber, a butyl rubber, fluoroelastomer, a perfluoroelastomer, a perfluoro polyether elastomer, or a thermoplastic elastomer including styrenic, polyether, polyester, polyurethane block copolymers, or combinations thereof.

Embodiment 34 is the composite tube of any of Embodiments 26-33, wherein the polyethylene layer includes expanded polyethylene.

Embodiment 35 is the composite tube of any of Embodiments 26-34, wherein the polyethylene layer includes expanded ultra-high molecular weight polyethylene.

Embodiment 36 is the composite tube of any of Embodiments 26-35, wherein the at least one elastomer forms a coating on the at least one polyethylene layer.

Embodiment 37 is the composite tube of any of Embodiments 26-36, wherein the composite tube is γ-sterilized, steam sterilized, autoclave sterilized, EtO sterilized, x-ray sterilized, e-beam sterilized, dry heat sterilized, or cleaned in place (CIP).

Embodiment 38 is the composite tube of any of Embodiments 26-37, wherein the tube is peristaltic pump tubing.

In another example (“Embodiment 39”), a peristaltic pump tubing includes a gamma sterilized polymeric tube having an average pump life from 300 to 5,000 hours.

In another example (“Embodiment 40”), a sterilized non-fluoropolymer tube having an average pump life from 300 to 5,000 hours.

The foregoing embodiments are just that and should not be read to limit or otherwise narrow the scope of any of the inventive concepts otherwise provided by the instant disclosure. While multiple embodiments are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a peristaltic pump having a pump tubing in accordance with an embodiment;

FIG. 2 is a cross-sectional view of a pump tubing in accordance with an embodiment; and

FIG. 3 is a differential scanning calorimetry (DSC) curve for a composite tube having a silicone elastomer reinforced with UHMWPE in accordance with an embodiment.

### DETAILED DESCRIPTION

### Definitions and Terminology

This disclosure is not meant to be read in a restrictive manner. For example, the terminology used in the application should be read broadly in the context of the meaning those in the field would attribute such terminology.

As used herein, the term “membrane” means a polymer in the form of an essentially two-dimensional sheet, wherein the length and the width are both much greater than the thickness, for example both the length and the width are at least 100 times the thickness. In some embodiments, the membrane is a microporous membrane having a structure that allows, for example, water vapor to pass through the thickness of the membrane without liquid water being able to penetrate from one side of the membrane to the other.

The term “film” means a membrane wherein the pores have been at least partially filled with a polymer such that the flow of gases or liquids does not occur through open pore channels in the membrane.

The term “porous” as used herein means the porosity of a membrane or layer is sufficient to allow penetration of an elastomer.

The term “on” as used herein is meant to describe an element directly on another element or indirectly on the other element with intervening elements present.

The singular forms “a”, “an”, and “the” as used herein include plural reference unless the context clearly dictates otherwise.

The term “pump life” as used herein is meant to describe the amount of time a tube can withstand use within a peristaltic pump before failure.

The term “wrap count” as used herein is meant to describe the number of layers of polymer (e.g., expanded polyethylene) within a tube wall.

The term “volume fraction” as used herein is meant to describe the proportion of polymer (e.g., expanded polyethylene) to elastomer (e.g., silicone) within a given tubing configuration stated in a percentage (%) of total volume.

The term “gamma irradiation” as used herein is meant to describe the sterilization technique where the tubes are subject to gamma radiation. Gamma radiation may be measured in the unit kilogray (kGy).

The term “ultra-high molecular weight polyethylene” and “(UHMWPE)” may be used interchangeably and as used herein are meant to describe a homopolymer of ethylene or a copolymer of ethylene and at least one comonomer (e.g., alpha olefins or cyclic olefins having 3 to 20 carbon atoms). Comonomers may be present in the UHMWPE in the copolymers in the amount from about 0.001 mol % to about 10 mol %. UHMWPE polymers have a weight average molecular weight (Mw) between about 500,000 g/mol and about 10,000,000 g/mol.

The term “differential scanning calorimetry (DSC) curve” as used herein is meant to describe a schematic curve showing the amount of energy (y) required to maintain each temperature (x) scanned across a range of temperatures.

The term “primary melt peak temperature” as used herein is meant to describe the peak temperature in the largest melt endotherm as defined by the area under the endotherm on a DSC curve.

The terms “about” and “approximately” can be understood to mean plus or minus 10% of the stated value.

### DESCRIPTION OF VARIOUS EMBODIMENTS

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale but may be exaggerated to illustrate various aspects of the present disclosure, and in that regard, the drawing figures should not be construed as limiting.

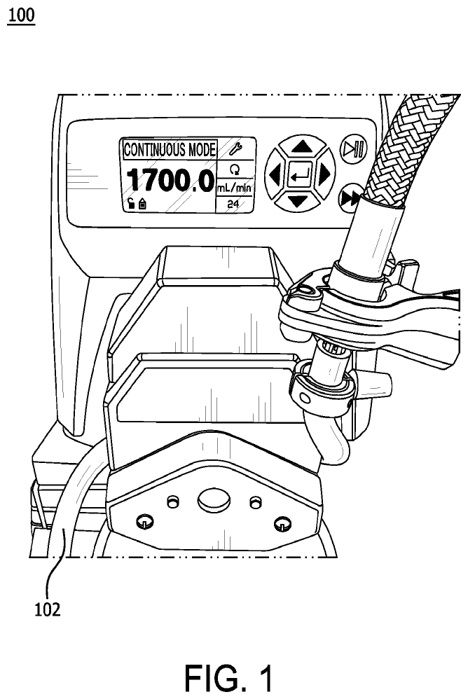

The present disclosure generally relates to a composite pump tubing having an elastomer reinforced with porous polyethylene. FIG. 1 is an embodiment of a peristaltic pump 100 having a pump tubing 102.

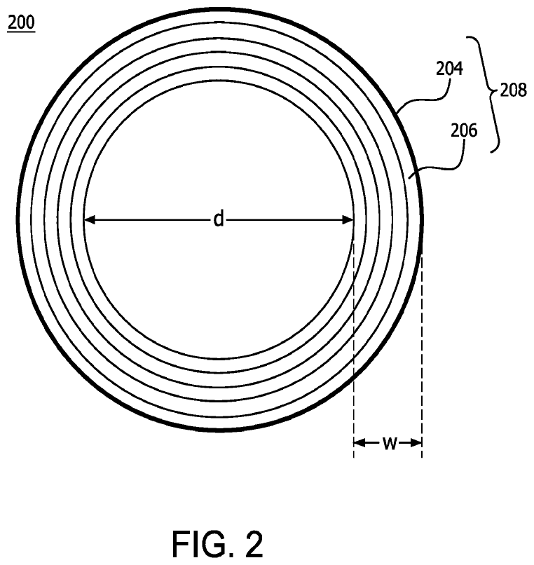

FIG. 2 is a cross-sectional view of a composite tube 200 in accordance with an embodiment. The composite tube 200 may be a pump tubing used in a peristaltic pump (e.g., the peristaltic pump shown in FIG. 1). In some instances, the composite tube 200 may be used in pinch valves. In some instances, for example as shown in FIG. 2, the composite tube 200 may have a concentric circular cross-sectional shape. In some instances, the composite tube 200 may have a spiral cross section.

In an embodiment, as shown in FIG. 2, a composite tube 200 includes a tube wall (w) having at least one porous polyethylene layer 204. Each porous polyethylene layer 204 may be imbibed with at least one elastomer 206 to form a composite layer 208 having an elastic modulus less than about 40 MPa and a primary melt peak temperature less than about 135° C. For example, the composite tube 200 as shown in FIG. 2 has a tube wall w including five composite layers 208. In some instances, the composite layer 208 may have a primary melt peak temperature greater than about 127° C. and below about 145° C. A secondary melt temperature higher than the primary melt peak temperature may be observed in oriented films (e.g., films that are stretched or oriented in one direction). The secondary melt temperature (e.g., associated with a second endotherm) may be from about 145° C. to about 155° C.

The composite layer 208 may be produced by any one of a variety of methods including gravure coating to impregnate the porous polyethylene layer with elastomer, for example, as discussed and shown in (U.S. Pat. No. 6,451,396 to Zumbrum, et al.). Optionally, the impregnated polyethylene layer (i.e., composite layer 208) can be conveyed to another roller for the application of a top coat of liquid elastomer. The amount of liquid elastomer impregnated into the polyethylene layer may be varied to produce composites of desired elastomer content. The coated structure is then either taken up in an uncured state around a cylindrical mandrel and wound to a desired wall thickness or passed through a convection oven to cure the liquid elastomer and form a polyethylene reinforced elastomer membrane. In the uncured state, the elastomer impregnated polyethylene may either be heated on a mandrel to form a tubular article, such as pump tubing (e.g., pump tubing 102 shown in FIG. 1). In some instances, the coated membrane may be taken up onto a mandrel and sliced into tapes of desired width. The tapes can then be wrapped around a mandrel using filament winding techniques to generate three dimensional objects of irregular shape and unlimited length.

In some embodiments, the composite tube 200 may have a volume fraction from about 1% to about 20% of the proportion of polymer to elastomer within a given tubing configuration stated in a percentage (%) of total volume. In some instances, the composite tube 200 may have a volume fraction from about 2% to about 18%, or from about 3% to about 15%, or from about 4% to about 14%, or from about 4% to about 13%. In one embodiment, the composite tube 200 may have a volume fraction from about 7% to about 13%. In yet another embodiment, the composite tube 200 may have a volume fraction from about 4% to about 7%.

In some examples, elastic modulus values of the composite layer 208 are related to volume fraction of reinforcing film based on UHMWPE. In some embodiments, the elastic modulus of the composite layer 208 may range from about 1 MPa to about 40 MPa, from about 1 MPa to about 35 MPa, from about 1 MPa to about 30 MPa, from about 1 MPa to about 25 MPa, from about 1 MPa to about 20 MPa, from about 1 MPa to about 15 MPa, from about 1 MPa to about 10 MPa, or from about 1 MPa to about 5 MPa. In some embodiments, the elastic modulus of the composite layer 208 may range from about 1 MPa to about 7 MPa, or from about 1 MPa to about 13 MPa, or from about 1 MPa to about 24 MPa. A Voight two phase composite model can be used to estimate the changes in elastic modulus of the composite layer 208 as a function of volume fraction of reinforcing film. Table 1 includes estimated upper and lower values of elastic modulus using Voight Composite model for ranges of different volume fraction tubes having an inner diameter of about 6.4 mm and a tube wall thickness of 2.4 mm. The Voight two phase composite model relates the Elastic Modulus of the composite layer (E_{c}) to the volume fractions of the elastomer (Vₑ) and reinforcing layer (V_{f}) and the Elastic Modulus of the elastomer (Eₑ) and Elastic Modulus of the reinforcing layer (E_{f}) as follows: E_{c}=E_{f}V_{f}+EₑVₑ.

TABLE 1

Estimated Elastic Modulus (MPa)

| Volume Fraction (%) | Upper | Lower |
|---|---|---|
| 1-3 | 8 | 1 |
| 3-7 | 11 | 1 |
| 7-13 | 24 | 1 |
| 13-17 | 31 | 1 |
| 17-20 | 37 | 1 |

In some examples, the porous polyethylene of the layer 204 may be expanded polyethylene (ePe). In some embodiments, the porous polyethylene may be expanded ultra-high molecular weight polyethylene (eUHMWPE). eUHMWPE polymers may have a primary melt peak temperature greater than about 127° C. and below about 145° C. A secondary melt temperature higher than the primary melt peak temperature may be observed in oriented films. For eUHMWPE, the secondary melt temperature (e.g., associated with the second endotherm) is from about 145° C. to about 155° C.

In some instances, the polyethylene forming the polyethylene layer 204 (hereafter “polyethylene”) may include a filler such as fumed silica, colloidal silica, carbon black, or combinations thereof. In some instances, the polyethylene may include a plasma treatment. In some instances, the polyethylene may optionally include a silane coupling agent. The polyethylene layer 204 may have a density of from about 0.05 g/cc to about 0.8 g/cc. In some embodiments, the layer 204 may have a density of from about 0.1 g/cc to about 0.7 g/cc, from about 0.2 to about 0.6 g/cc, from about 0.3 to about 0.5 g/cc, or from about 0.3 to about 0.4 g/cc.

In some embodiments, the composite tube 200 may have a wall thickness (i.e., the thickness of the tube wall (w)) of from about 0.5 to about 10 mm. In some embodiments, the composite tube 200 may have a wall thickness of from about 0.9 mm to about 9.2 mm, from about 1 mm to about 8 mm, from about 1.1 mm to about 7 mm, from about 1.2 mm to about 6 mm, from about 1.3 mm to about 5.5 mm, or from about 1.4 mm to about 5 mm. In one embodiment, the composite tube 200 may have a wall thickness of from about 1.5 mm to about 4.9 mm. Each of the composite layers may have a thickness of from about 0.3 mm to about 10 mm. The ratio of wall thickness to inside diameter of the composite tube may be less than about 2 mm, less than about 1.9 mm, less than about 1.8 mm, less than about 1.7 mm, or less than about 1.6 mm. A wrap count of layers 204 within the composite tube 200 may be from about 1 to about 200, or from about 1 to about 190, or from about 1 to about 185, or from about 1 to about 180, or from 1 to about 175. In some embodiments, the wrap count of layers 204 within the composite tube 200 may be from about 1 to about 170.

The composite tube 200 may include a reinforcing film including, but not limited to nonwovens, extruded and cast films, expanded membranes, and phase inversion membranes. Suitable porosity may be included through mechanical methods if not inherent in the as produced reinforcing film. Polymers suitable for use as a reinforcing film include but are not limited to polyethylene, polypropylene, polyether ketone) (PEEK), and copolymers of ethylene and at least one comonomer. Suitable comonomers that may be used include but are not limited to alpha olefins or cyclic olefins having 3 to 20 carbon atoms.

In some examples, the composite tube 200 has an average pump life of between about 80 hours to about 5000 hours. In some examples, the composite tube 200 has an average pump life of between about 88 hours to about 5000 hours. In some embodiments, the composite tube 200 has an average pump life of between about 200 hours to about 4000 hours. In some embodiments, the composite tube 200 has an average pump life of between about 250 hours to about 3000 hours. In some embodiments, the composite tube 200 has an average pump life of between about 300 hours to about 3000 hours. In some embodiments, the composite tube 200 has an average pump life of from about 100 hours to about 350 hours when subject to gamma radiation of about 50 kGy. In some embodiments, the composite tube 200 has an average pump life of from about 100 hours to about 320 hours when subject to gamma radiation of about 50 kGy. In some instances, the gamma radiation may be higher than 50 kGy. In some embodiments, the composite tube 200 may be subject to gamma radiation from about 20 kGy to about 60 kGy, or from about 20 kGy to about 50 kGy, or from about 20 kGy to about 40 kGy. In some embodiments, the composite tube 200 may be subject to gamma radiation from about 20 kGy to about 30 kGy. In some instances, the composite tube 200 may be subject to gamma radiation more than once.

In some examples, the at least one elastomer 206 may be at least partially imbibed through a thickness of the porous polyethylene layer 204, and the at least one elastomer 206 forms a coating on at least one polyethylene layer 204. In some embodiments, for example, the at least one elastomer 206 may be from about 1% to about 100% imbibed through a polyethylene layer 204. In some embodiments, the at least one elastomer 206 may be from about 5% to less than or equal to 100%, from about 10% to less than or equal to 100%, from about 15% to less than or equal to 100%, or from about 20% to less than or equal to 100% imbibed through a polyethylene layer 204.

The elastomer 206 may be selected from, but not limited to, thermosetting and thermoplastic elastomers. The elastomer 206 may be solvated or used neat for processing into a tube. Thermosetting elastomers include diene-based rubbers such as natural rubber, styrene-butadiene rubber (SBR), isoprene rubber (IR), butadiene rubber (BR), chloroprene rubber (CR), and acrylonitrile-butadiene rubber (NBR), and butyl rubber (isobutylene/isoprene rubber (IIR)), ethylene/propylene rubber (EPM), ethylene/propylene/diene rubber (EPDM), urethane resin, silicone rubber, fluoroelastomer (FKM), perfluorofluoroelastomer (FFKM), fluorinated silicone rubber and perfluoroether elastomers. Thermoplastic elastomers (TPE) may include styrenic, polyether, polyester, polyurethane block copolymers.

The elastomer 206 may include a silicone (e.g., methyl silicone, phenyl silicone, fluorosilicone, etc.). In some instances, the elastomer 206 may include an organosilicone. In some instances, the elastomer 206 may include a methyl silicone, a phenyl silicone, a fluorosilicone, or combinations thereof. In some instances, the elastomer 206 may include a fluoroelastomer, a perfluoroelastomer, a perfluoro polyether elastomer, or a thermoplastic elastomer including styrenic, polyether, polyester, polyurethane block copolymers, or combinations thereof.

In some examples, the composite tube 200 may be sterilized. For example, the composite tube 200 may be sterilized by gamma (γ) radiation, steam, steam in place (SIP), clean in place (CIP), autoclave, e-beam, x-ray, dry heat, and ethylene oxide (EtO). In one embodiment, the composite tube 200 may be sterilized by gamma (γ) radiation.

In some embodiments, a peristaltic pump tubing includes at least one porous polymeric layer, each porous polymeric layer being imbibed with at least one elastomer to form a composite layer having an elastic modulus less than 40 MPa. In some examples, a gamma sterilized polymeric tube has an average pump life of from about 24 hours to about 1950 hours, from about 25 hours to about 1900 hours, from about 26 hours to about 1850 hours, from about 27 hours to about 1830 hours, from about 28 hours to about 1810 hours, or from about 29 hours to about 1800 hours. In some examples, a gamma sterilized polymeric tube has an average pump life of from about 30 hours to about 1780 hours.

In some embodiments, a composite tube includes a tube wall having at least one polyethylene layer, each polyethylene layer being coated with at least one elastomer to form a composite layer having an elastic modulus less than about 11 MPa and a primary melt peak temperature less than 135° C. In some instances, the composite layer may have a primary melt peak temperature greater than about 127° C. and below about 145° C. A second higher temperature endotherm may be observed in oriented films. The second endotherm is from about 145° C. to about 155° C. In some examples, the composite tube has an average pump life greater than about 190 hours. In some examples, at least one elastomer forms a coating on at least one polyethylene layer.

In some embodiments, a peristaltic pump tubing includes a gamma sterilized polymeric tube having an average pump life from about 80 hours to about 5000 hours, or from about 83 hours to about 4500 hours, or from about 85 hours to about 4000 hours, or from about 88 hours to about 3500 hours. In some embodiments, the gamma sterilized polymeric tube may have an average pump life from about 90 hours to about 5000 hours, or from about 100 hours to about 4800 hours, or from about 110 hours to about 4600 hours, or from about 120 hours to about 4400 hours, or from about 130 hours to about 4200 hours, or from about 140 hours to about 4100 hours, or from 150 hours to about 4000 hours. In some embodiments, the gamma sterilized polymeric tube may have an average pump life from about 300 to about 3000 hours.

In some embodiments, a sterilized non-fluoropolymer tube may have an average pump life from 80 to 5,000 hours, or from about 83 hours to about 4500 hours, or from about 85 hours to about 4000 hours, or from about 88 hours to about 3500 hours. In some embodiments, the sterilizable non-fluoropolymer tube may have an average pump life from about 90 hours to about 5000 hours, or from about 100 hours to about 4800 hours, or from about 110 hours to about 4600 hours, or from about 120 hours to about 4400 hours, or from about 130 hours to about 4200 hours, or from about 140 hours to about 4100 hours, or from 150 hours to about 4000 hours. In some embodiments, the sterilizable non-fluoropolymer tube may have an average pump life from about 300 to about 3000 hours.

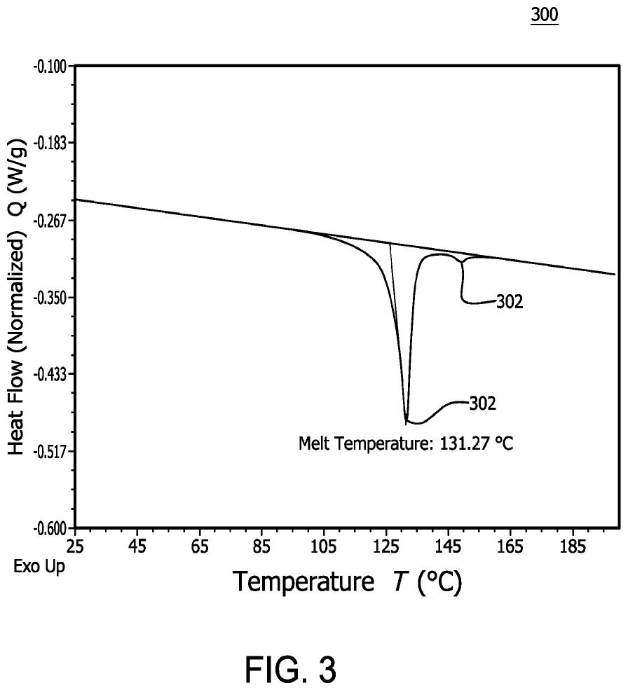

FIG. 3 is a schematic differential scanning calorimetry (DSC) curve 300 an ultra-high molecular weight polyethylene tube in accordance with an embodiment. UHMWPE polymers have a primary melt peak temperature (e.g., corresponding to a first endotherm) greater than about 127° C. and below about 145° C. In some embodiments, for example as shown in FIG. 3, a UHMWPE polymer may have a primary melt peak temperature at a first peak 302 at 131° C. In some embodiments, a secondary melt temperature (e.g., corresponding to a second endotherm) is shown at a second peak 304 in FIG. 3, and is from about 145° C. to about 155° C.

### Test Methods

### Pump Life

A sample tube was cut to a length between 152 mm and 356 mm using a standard tube cutter. The tube was outfitted with a single high polish quick-clamp sanitary tube fitting (appropriate size of fitting is dependent on the desired tube to be tested) that was held down with a single shaft collar on one end of the tube. The shaft collar was tightened with an Allen wrench until finger tight. The tube was then positioned into a pump drive (Masterflex model 07551-20) equipped with a pump head (Masterflex model 77200-52). The pump tube was centered in the pump head. The end of the tube was outfitted with quick clamp connection and shaft collar was positioned towards the outlet of flow for the setup. For counterclockwise rotation, the shaft collar was positioned on the right side of pump head before closing the pump head fully. For clockwise rotation position, the shaft collar was placed on the left side of pump head before closing the pump head fully. The pump drive was then set to following settings: Continuous Flow, 1700 RPM, Tube Size ## (with the number being set to the appropriate size for the desired tube to be tested).

The fluid reservoir (Nalgene model 2015-2000) was filled with deionized water to a value greater than 1800 mL. The cap (Nalgene model 2135-5302) was administered back onto the reservoir. One of the tubes from the cap of the fluid reservoir was connected to the side of the sample tube not outfitted with the quick clamp connection using a reducer barb mender/splicer.

The sample tube side outfitted with the quick clamp connection was connected to a flow path using a quick clamp tube connector. The flow path contained the following components that are listed in the order of their placement within the flow path: Outlet tube with quick clamp connector, Stainless Steel CheckValve (McMaster-Carr model 1874N13 commercially available from McMaster-Carr), pressure sensor (GEMS SENSORS model 2200BGG1002A3UA), pressure regulator (Go regulator model BP3-2A41151111), flow meter (IFM efector model SM600) with mounting adapter (IFM efector model E40200) and then attached back to the fluid reservoir using reducer barb mender/splicer. The pressure sensor was used to measure the pressure within the setup throughout the duration of testing and was collected with WONDERWARE software (sampling rate 1 measurement every 30 seconds). The flow within the setup was measured throughout the duration of the testing using the flow meter and was collected by WONDERWARE software (sampling rate 1 measurement every 30 seconds). The pump drive was then turned on and the pressure knob was used to set a pressure of 20 psi within the system. A sample was said to have failed the test under two circumstances.

The first circumstance was rupture of the tube in which any visible liquid water leakage was observed flowing out of the pump head. The second circumstance was flow decay in which flow rate data throughout testing was analyzed. Flow rate data was analyzed by recording the initial flow rate value of the sample 2 minutes after the pressure was set to its desired value within the system. This initial flow value was then compared to the average flow rate for the last 30 minutes of testing that has been conducted (e.g., a time of 1 hour and 3 minutes will report an average flow rate value calculated by taking the average of all flow rate data collected from 33 minutes to 1 hour and 3 minutes). If this average flow rate value ever dropped below 75% of the initial flow value, the sample was determined to have failed the test and testing is stopped. The accumulated time from the start time of testing to time of rupture or time of flow decay >25% of the tube was reported in hours.

### Differential Scanning Calorimetry (DSC) and Primary Melt Peak Temperature

Differential Scanning calorimetry data was collected using a TA Instruments (159 Lukens Drive, New Castle, DE 19720) Q2000 DSC using TZero Aluminum Pans and lids. Scans were run from 25° C. to 200° C. at 10° C./min. A plug was cut from the side wall of tubes using a 2 mm diameter biopsy punch. The plug was then sectioned to an approximately 1 mm thickness×2 mm thickness diameter puck. The primary melt peak temperature was taken as the peak in the melt endotherm centered below 145° C.

### Elastic Modulus

The elastic modulus of the composite layer (e.g., the composite layer 208 as shown in FIG. 2) was determined from layers peeled from the tube. Composite layers were peeled from tubes by inserting a blunt tweezer between the composite layers and slit along the long axis of the tube to initiate peel. Sufficient length was peeled to obtain approximately a single composite layer. Tensile specimens were cut from the peeled layer using an ASTM D638 Type V Dog bone die in the hoop direction (direction of peel). The tensile behavior was measured on an Instron® Model 5564 (Illinois Tool Works Inc, Norwood MA) equipped with flat-faced grips and a 100 N load cell. Distance between grips was 25.4 mm with a gauge length of 7.62 mm was tested at a crosshead speed of 1.27 mm/sec (16.6%/sec). Elastic modulus was calculated from the initial linear portion of engineering stress (Load/Area)/engineering strain (change in length/gauge length) curve. A minimum of two samples were tested and the elastic modulus reported is an average of the individual values.

### Thickness Measurement

Samples thickness for the peeled layers were measured using a Mitotoyo Absolute Digital Micrometer Model ID-C112E (Mitutoyo America Corporation, Aurora IL) using a 6.35 mm diameter flat probe. Three measurements were taken, and the average thickness was reported.

### Volume Fraction

The volume fraction of silicone was calculated by adding the thickness of the silicone top-coat layer (i.e., the silicone that does not enter within the membrane pores but sits on top of the membrane) to the thickness of silicone in the impregnated layer (e.g., layer 208 as shown in FIG. 2), where the impregnated layer is the layer of material that encompasses ePe that has silicone that fills its pores. The silicone content in the impregnated layer was calculated from the ratio of expanded polyethylene density to polyethylene density which was then divided by the total thickness of the composite.

In some embodiments, the silicone top-coat thickness may be about 0.056 mm, the density ratio (0.16 g/cc/0.94 g/cc) was 0.17 which yielded a silicone content of 0.02 mm in the impregnated layer, which when divided by the total thickness of 0.08 mm resulted in a volume % of 5.1.

### EXAMPLES

### Example 1

A peristaltic pump tubing (“Sample I”) (d=6.4 mm and w=2.4 mm) was prepared by first obtaining an UHMWPE membrane that was the same in composition as defined by U.S. Pat. No. 10,577,468 to Sbriglia. The UHMWPE membrane was passed between a gravure roll and a silicone rubber roll and then passed through two chrome rolls having a 3.5 mil gap therebetween containing liquid silicone as described in U.S. Pat. No. 6,451,396 to Zumbrum, et al. The UHMWPE membrane was coated at a speed of 2 feet per minute and taken up onto a mandrel having an outer diameter of 6.4 mm (i.e., the inner diameter of the composite tube). A composite tube having a wall thickness of 2.4 mm was obtained.

The uncured composite tube was then placed in a press at 135° C. for 12 minutes at a pressure of 20 tonnes and removed from the mandrel. The pump tubing was post-baked for 6 hours at 110° C. to bring about a final cure and to remove any volatiles. Additional processing in the form of heat treatment was then completed such as described by U.S. Patent Publication No. 2021/0317276 to Bell, et al. The pump tubing was then gamma sterilized at a value from 29 kGY to 34 kGY. The subsequent volume fraction of the tube produced was determined to be 6%.

The composite elastomer tubing of Sample 1 was then tested in accordance with the pump life test method described above. The composite elastomer tubing of Sample 1 ruptured at approximately 1781 hrs (n=2, max=1942 hrs). An elastic modulus of 9.8 MPa was determined for Sample I. A melt peak temperature of Sample 1 was determined to be 132.7° C. The data is set forth in Table 2.

### Example 2

A peristaltic pump tubing (“Sample II”) (d=6.4 mm and w=2.4 mm) was prepared by first obtaining an UHMWPE membrane that was the same in composition as defined by U.S. Pat. No. 10,577,468 to Sbriglia. The UHMWPE membrane was passed between a gravure roll and a silicone rubber roll and then passed through two chrome rolls having a 3.5 mil gap therebetween containing liquid silicone as described in U.S. Pat. No. 6,451,396 to Zumbrum, et al. The UHMWPE membrane was coated at a speed of 2 feet per minute and taken up onto a mandrel having an outer diameter of 6.4 mm (i.e., the inner diameter of the composite tube.) A wall thickness of 2.4 mm was obtained.

Next, the uncured composite was placed in a press at 135° C. for 12 minutes at a pressure of 20 tonnes and removed from the mandrel. The pump tubing was baked for 6 hours at 110° C. to bring about a final cure and remove volatiles. The pump tubing was then gamma sterilized at a value from 29 kGY to 34 kGY. The subsequent volume fraction of the tube produced was determined to be 6%.

The composite elastomer tubing of Sample II was then tested in accordance with the pump life test method described above. The composite elastomer tubing of Sample II ruptured at approximately 24 hrs (n=2, max=26 hrs). An elastic modulus of 22.5 MPa was determined for Sample II. A melt peak temperature of Sample II was determined to be 138° C. The data is set forth in Table 2.

### Example 3

A peristaltic pump tubing (“Sample III”) (d=6.4 mm and w=2.4 mm) was prepared by first obtaining an ePe membrane (3P07A membrane from W.L. Gore & Associates, Inc., Heerlen, NL). The ePe membrane was 0.016 mm thick, 914 mm wide, and was obtained as a continuous roll. The ePe membrane had a density of 0.16 g/cc. The ePe membrane was passed between a gravure roll and a silicone rubber roll and then passed through two chrome rolls having a 3.5 mil gap therebetween containing liquid silicone as described in U.S. Pat. No. 6,451,396 to Zumbrum, et al. The ePe membrane was coated at a speed of 1 foot per minute and taken up onto a mandrel having an outer diameter of 6.4 mm (i.e., the inner diameter of the composite tube). A wall thickness of 2.4 mm was obtained.

Next, the uncured composite on the mandrel was placed in a press at 135° C. for 12 minutes at a pressure of 20 tonnes and then removed from the mandrel. The pump tubing was post-baked for 6 hours at 110° C. to bring about a final cure and remove all volatiles. Additional processing in the form of heat treatment, such as is described by U.S. Patent Publication No. 2021/0317276 to Bell, et al. was conducted. The pump tubing then gamma sterilized at a value from 45 kGY to 51 kGY. The subsequent volume fraction of the tube produced was determined to be 4%.

The composite elastomer tubing of Sample III was then tested in accordance with the pump life test method described above. The composite elastomer tubing of Sample III ruptured at approximately 1011 hrs (n=2, max=1031 hrs). An elastic modulus of 2.9 MPa was determined for Sample III. A melt peak temperature of Sample III was determined to be 131.8° C. The data is set forth in Table 2.

### Example 4

A peristaltic pump tubing (“Sample IV”) (d=6.4 mm and w=2.4 mm) was prepared by first obtaining an ePe membrane (3P07A membrane from W.L. Gore & Associates, Inc., Heerlen, NL). The ePe membrane had a thickness of 0.016 mm, a width of 914 mm, and was obtained as a continuous roll. The ePe membrane had a density of 0.16 g/cc. The ePe membrane was passed between a gravure roll and a silicone rubber roll and then passed through two chrome rolls having a 3.5 mil gap therebetween containing liquid silicone as described in U.S. Pat. No. 6,451,396 to Zumbrum, et al. The ePe membrane was coated at a speed of 1 foot per minute and taken up onto a mandrel having an outer diameter of 6.4 mm (i.e., the inner diameter of the composite tube). A composite tube having a wall thickness of 2.4 mm was obtained.

Next, the uncured composite was placed in a press at 135° C. for 12 minutes at a pressure of 20 tonnes and then removed from the mandrel. The pump tubing was baked for 6 hours at 110° C. to bring about a final cure and remove any volatiles. The pump tubing was then gamma sterilized at a value from 45 kGY to 51 kGY. The subsequent volume fraction of the tube produced was determined to be 4%.

The composite elastomer tubing of Sample IV was then tested in accordance with the pump life test method described above. The composite elastomer tubing of Sample IV ruptured at approximately 32 hrs (n=2, max=53 hrs). An elastic modulus of 7.8 MPa was determined for Sample IV. A melt peak temperature of Sample IV was determined to be 140.9° C. The data is set forth in Table 2.

### Example 5

A peristaltic pump tubing (“Sample V”) (d=6.4 mm and w=2.4 mm) was prepared by first obtaining an ePe membrane (3P07A membrane from W.L. Gore & Associates, Inc., Heerlen, NL). The ePe membrane had a thickness of 0.016 mm, a width of 762 mm, and was obtained as a continuous roll. The ePe membrane had a density of 0.16 g/cc. The ePe membrane was passed between a gravure roll and a silicone rubber roll and then passed through two chrome rolls having a 1 mil gap therebetween containing liquid silicone as described in U.S. Pat. No. 6,451,396 to Zumbrum, et al. The ePe membrane was coated at a speed of 3 foot per minute and taken up onto a mandrel having an outer diameter of 6.4 mm (i.e., the inner diameter of the composite tube). A composite tube having a wall thickness of 2.4 mm was obtained.

Next, the uncured composite was placed in an oven at 135° C. for 24 minutes and then removed from the mandrel. The pump tubing was baked for 6 hours at 110° C. to bring about a final cure and remove any volatiles. Additional processing in the form of heat treatment, such as is described by U.S. Patent Publication No. 2021/0317276 to Bell, et al. was conducted. The pump tubing was then gamma sterilized at a value from 45 kGY to 51 kGY. The subsequent volume fraction of the tube produced was determined to be 11%.

The composite elastomer tubing of Sample V was then tested in accordance with the pump life test method described above. The composite elastomer tubing of Sample V ruptured at approximately 474 hrs (n=2, max=936 hrs). An elastic modulus of 13.3 MPa was determined for Sample V. A melt peak temperature of Sample V was determined to be 133.2° C. The data is set forth in Table 2.

TABLE 2

| | | | | | Average | Composite | |
|---|---|---|---|---|---|---|---|
| | Inner | Volume | Gamma | Additional | Pump | layer Elastic | Melt |
| | Diameter | Fraction | Dosage | Heat | Life | Tensile Modulus | Temperature |
| Sample | (mm) | (%) | (kGy) | Treatment | (Hrs.) | (MPa) | (° C.) |
| I | 6.4 | 6 | 29-34 | Yes | 1781 | 9.8 | 132.7 |
| II | 6.4 | 6 | 29-34 | No | 24 | 22.5 | 138 |
| III | 6.4 | 4 | 45-51 | Yes | 1011 | 2.9 | 131.8 |
| IV | 6.4 | 4 | 45-51 | No | 32 | 7.8 | 140.9 |
| V | 6.4 | 11 | 45-51 | Yes | 474 | 13.3 | 133.2 |

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A composite tube comprising:
a tube wall having at least one porous polyethylene layer, each said porous polyethylene layer being imbibed with at least one elastomer to form a composite layer having an elastic modulus less than 40 MPa and a primary melt peak temperature less than 135° C.

2. The composite tube of claim 1, wherein the composite tube has an average pump life greater than 80 hours.

3. The composite tube of claim 1, wherein the tube wall has a volume fraction from 1% to 20%.

4. The composite tube of claim 3, wherein the composite layer has an elastic modulus from about 1 MPa to about 40 MPa.

5. (canceled)

6. (canceled)

7. (canceled)

8. The composite tube of claim 1, wherein the porous polyethylene is expanded polyethylene (ePe).

9. The composite tube of claim 1, wherein the porous polyethylene is expanded ultra-high molecular weight polyethylene.

10. The composite tube of claim 1, wherein the elastomer is selected from a silicone, a diene-based rubber, a butyl rubber, fluoroelastomer, a perfluoroelastomer, a perfluoro polyether elastomer, or a thermoplastic elastomer including styrenic, polyether, polyester, polyurethane block copolymers, or combinations thereof.

11. The composite tube of claim 1, wherein the at least one elastomer is at least partially imbibed through a thickness of the porous polyethylene layer.

12. The composite tube of claim 1, wherein the composite tube is γ-sterilized, steam sterilized, autoclave sterilized, EtO sterilized, x-ray sterilized, e-beam sterilized, dry heat sterilized, or cleaned in place (CIP).

13. The composite tube of claim 1, wherein the tube is peristaltic pump tubing.

14. A peristaltic pump tubing comprising:
a tube wall having at least one porous polymeric layer, each said porous polymeric layer being imbibed with at least one elastomer to form a composite layer having an elastic modulus less than 40 MPa;
wherein the peristaltic pump tubing has an average pump life from 300 to 5,000 hours.

15. The pump tubing of claim 14, wherein the composite layer has a primary melt peak temperature less than 135° C.

16. The pump tubing of claim 14, wherein the tube wall has a volume fraction from 1% to 13%.

17. The pump tubing of claim 16, wherein the composite layer has an elastic modulus from about 1 MPa to about 40 MPa.

18. (canceled)

19. (canceled)

20. (canceled)

21. The pump tubing of claim 14, wherein the peristaltic pump tubing is γ-sterilized, steam sterilized, autoclave sterilized, EtO sterilized, x-ray sterilized, e-beam sterilized, dry heat sterilized, or cleaned in place (CIP).

22. The pump tubing of claim 14, wherein the at least one elastomer is at least partially imbibed through a thickness of the porous polyethylene layer.

23. The pump tubing of claim 14, wherein the elastomer is selected from a silicone, a diene-based rubber, a butyl rubber, fluoroelastomer, a perfluoroelastomer, a perfluoro polyether elastomer, or a thermoplastic elastomer including styrenic, polyether, polyester, polyurethane block copolymers, or combinations thereof.

24. The pump tubing of claim 14, wherein the at least one porous polymeric layer is selected from porous polyethylene, polypropylene, poly(ether ketone), and copolymers of ethylene and at least one comonomer.

25. The pump tubing of claim 14, wherein the at least one porous polymeric layer comprises at least one of expanded polyethylene and ultra-high molecular weight polyethylene.

26. A composite tube comprising:
a tube wall having at least one polyethylene layer, each said polyethylene layer being coated with at least one elastomer to form a composite layer having an elastic modulus less than 40 MPa and a primary melt peak temperature less than 135° C.

27. The composite tube of claim 26, wherein the composite tube has an average pump life greater than about 80 hours.

28. The composite tube of claim 26, wherein the tube wall has a volume fraction from 1% to 20%.

29. The composite tube of claim 28, wherein the composite layer has an elastic modulus from about 1 MPa to about 40 MPa.

30. (canceled)

31. (canceled)

32. (canceled)

33. The composite tube of claim 26, wherein the elastomer is selected from a silicone, a diene-based rubber, a butyl rubber, fluoroelastomer, a perfluoroelastomer, a perfluoro polyether elastomer, or a thermoplastic elastomer including styrenic, polyether, polyester, polyurethane block copolymers, or combinations thereof.

34. The composite tube of claim 26, wherein the polyethylene layer comprises expanded polyethylene.

35. The composite tube of claim 26, wherein the polyethylene layer comprises expanded ultra-high molecular weight polyethylene.

36. The composite tube of claim 26, wherein the at least one elastomer forms a coating on the at least one polyethylene layer.

37. The composite tube of claim 26, wherein the composite tube is γ-sterilized, steam sterilized, autoclave sterilized, EtO sterilized, x-ray sterilized, e-beam sterilized, dry heat sterilized, or cleaned in place (CIP).

38. The composite tube of claim 26, wherein the tube is peristaltic pump tubing.

39. (canceled)

40. (canceled)
